(19) United States
(12) Patent Application Publication
(10) Pub. No.: US 2021/0121716 A1
(43) Pub. Date: Apr. 29, 2021

(54) MONITORING SYSTEM FOR RADIATION THERAPY CENTER

(71) Applicant: OUR UNITED CORPORATION, Shaanxi (CN)

(72) Inventors: Jinsheng Li, Shaanxi (CN); Hongbin Zhao, Shaanxi (CN); Hao Yan, Shaanxi (CN)

(21) Appl. No.: 15/734,217

(22) PCT Filed: May 31, 2019
(86) PCT No.: PCT/CN2019/089710

§ 371 Date: Dec. 1, 2020

(30) Foreign Application Priority Data
Aug. 28, 2018 (CN) 201810990792.X

(51) Int. Cl. A61N 5/10; G16H 40/67

(52) U.S. Cl. CPC A61N 5/1064; G16H 40/67; A61N 5/1075; A61N 5/103

(57) ABSTRACT

Embodiments of the present disclosure provide a monitoring system for a radiotherapy center. The system includes at least one radiotherapy center and a monitoring cloud platform. The monitoring cloud platform is connected to the at least one radiotherapy center, and is configured to obtain operating parameters of at least one target device in the at least one radiotherapy center in real time. The monitoring cloud platform is further configured to control the at least one target device according to the operating parameters of the at least one target device in the at least one radiotherapy center.

### CROSS-REFERENCE TO RELATED APPLICATIONS

This application is a national phase entry under 35 USC 371 of International Patent Application No. PCT/CN2019/089710 filed on May 31, 2019, which claims priority to Chinese Patent Application No. 201810990792.X, filed with the Chinese Patent Office on Aug. 28, 2018, titled “MONITORING SYSTEM FOR RADIOTHERAPY CENTER”, which are incorporated herein by reference in their entirety.

### TECHNICAL FIELD

The present disclosure relates to the monitoring field, and in particular, to a monitoring system for a radiotherapy center.

### BACKGROUND

At present, a radiotherapy planning system (TPS) server in a radiotherapy center sends treatment parameters prestored in the TPS to a radiotherapy control system, and the radiotherapy control system sends the treatment parameters prestored to a radiotherapy device. The radiotherapy device treats a patient according to the treatment parameters prestored, and feeds back actual treatment parameters to the radiotherapy control system during a treatment process. The radiotherapy control system compares the actual treatment parameters with the treatment parameters prestored to achieve monitoring on the radiotherapy device.

### SUMMARY

Embodiments of the present disclosure provide a monitoring system for a radiotherapy center, which is configured to monitor operations of all devices of radiotherapy center(s), and may detect a failure of any device in the radiotherapy center(s) in time.

In order to achieve the above purpose, the technical solutions in the embodiments of the present disclosure are adopted.

In one aspect, a monitoring system for a radiotherapy center is provided. The monitoring system for the radiotherapy center includes at least one radiotherapy center and a monitoring cloud platform. The monitoring cloud platform is connected to the at least one radiotherapy center, and is configured to obtain operating parameters of at least one target device in the at least one radiotherapy center in real time. The monitoring cloud platform is further configured to control the at least one target device according to the operating parameters of the at least one target device in the at least one radiotherapy center.

In another aspect, a monitoring system for another radiotherapy center is provided. The monitoring system for the radiotherapy center includes at least one radiotherapy center and a monitoring cloud platform. The monitoring cloud platform is connected to the at least one radiotherapy center, and is configured to obtain patient treatment information generated by at least one target device in the at least one radiotherapy center in real time. The monitoring cloud platform is further configured to control the at least one target device according to the patient treatment information generated by the at least one target device in the at least one radiotherapy center.

In yet another aspect, a monitoring system for yet another radiotherapy center is provided. The monitoring system for the radiotherapy center includes at least one radiotherapy center and a monitoring cloud platform. The monitoring cloud platform is connected to the at least one radiotherapy center, and is configured to obtain operating parameters of at least one target device in the at least one radiotherapy center and patient treatment information generated by the at least one target device in real time. The monitoring cloud platform is further configured to control the at least one target device according to the operating parameters of the at least one target device and the patient treatment information generated by the at least one target device.

The monitoring system for the radiotherapy center provided by embodiments of the present disclosure includes at least one radiotherapy center and the monitoring cloud platform. The monitoring cloud platform is connected to the at least one radiotherapy center to obtain the operating parameters of the at least one target device in the at least one radiotherapy center and/or the patient treatment information generated by the at least one target device in real time. Then, the monitoring cloud platform controls the at least one target device according to the operating parameters of the at least one target device in the at least one radiotherapy center. The monitoring cloud platform may obtain the operating parameters of the at least one target device in the at least one radiotherapy center and/or the patient treatment information generated by the at least one target device in real time, and may also control the at least one target device according to the operating parameters of the at least one target device and/or the patient treatment information generated by the at least one target device. Therefore, the monitoring system for the radiotherapy center directly controls at least one corresponding device according to the operating parameters obtained, thereby monitoring all devices of the at least one radiotherapy center and monitoring processes of treating all patients. Further, potential safety hazards or treatment errors may be discovered in time when occurring in the at least one radiotherapy center, and corresponding remedial measures may be carried out.

### BRIEF DESCRIPTION OF THE DRAWINGS

In order to more clearly illustrate embodiments of the present disclosure or technical solutions in the prior art, drawings used in the description of the embodiments or the prior art will be briefly described below. Obviously, the drawings in the following description are merely some embodiments of the present disclosure, and a person skilled in the art can obtain other drawings according to these drawings without any creative effort.

FIG. 1 is a schematic diagram showing a structure of a monitoring system for a radiotherapy center, according to embodiments of the present disclosure; and

FIG. 2 is a schematic diagram showing a structure of another monitoring system for the radiotherapy center, according to embodiments of the present disclosure.

### DETAILED DESCRIPTION

Technical solutions in embodiments of the present disclosure will be described clearly and completely in combination with accompanying drawings in the embodiments of the present disclosure. Obviously, the described embodiments are merely some but not all of the embodiments of the present disclosure. All other embodiments obtained by a person of ordinary skill in the art based on the embodiments of the present disclosure without paying any creative effort shall be included in the protection scope of the present disclosure.

It will be noted that, phrase “for example” or “such as” is used to indicate an example, an illustration, or a description in the embodiments of the present disclosure. Any embodiment or design described as “for example” or “such as” in the embodiments of the present disclosure should not be construed as preferred or advantageous over other embodiments or designs. Rather, a use of the phrase “for example” or “such as” is intended to present a related concept in a concrete manner.

It will also be noted that, in the embodiments of the present disclosure, terms “of”, “corresponding” and “relevant” may sometimes be used in a mixed manner. It will be noted that, in a case where a difference among the terms is not emphasized, meanings of the terms to be expressed are the same.

In the related art, among respective devices of a radiotherapy center, only a working state of a radiotherapy device is monitored by a radiotherapy control system, and operating condition of remaining devices are not monitored. In a case where there are problems with working states of the remaining devices, the problems cannot be found in time, which easily leads to an occurrence of a potential safety hazard.

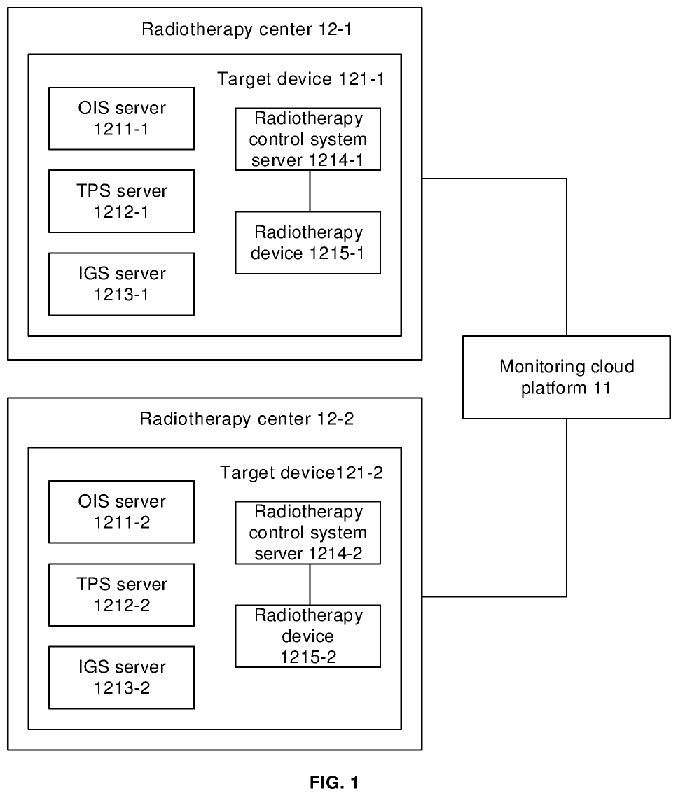

As for the above problem, referring to FIG. 1, embodiments of the present disclosure provide a monitoring system for a radiotherapy center, which includes at least one radiotherapy center 12 and a monitoring cloud platform 11.

The monitoring cloud platform 11 is connected to the at least one radiotherapy center 12 (two radiotherapy centers 12 are taken as an example in FIG. 1, and the two radiotherapy centers 12 are respectively marked with 12-1 and 12-2), and is configured to obtain operating parameters of target device(s) 121 (the target device 121 located in the radiotherapy center 12-1 in FIG. 1 is marked with 121-1, and the target device 121 located in the radiotherapy center 12-2 in FIG. 1 is marked with 121-2) in each radiotherapy center 12 and/or patient treatment information generated by the target device(s) 121.

For example, referring to FIG. 1, the target device(s) 121 may be one or more of the following (FIG. 1 shows an example in which the radiotherapy center(s) 12 include all existing target devices): tumor information system (OIS) server(s) 1211 (the OIS server located in the radiotherapy center 12-1 in FIG. 1 is marked with 1211-1, and the OIS server located in the radiotherapy center 12-2 is marked with 1211-2), treatment planning system (TPS) server(s) 1212 (the TPS server located in the radiotherapy center 12-1 in FIG. 1 is marked with 1212-1, and the TPS server located in the radiotherapy center 12-2 in FIG. 1 is marked with 1212-2), image guidance system (IGS) server(s) 1213 (the IGS server located in the radiotherapy center 12-1 in FIG. 1 is marked with 1213-1, and the IGS server located in the radiotherapy center 12-2 is marked with 1213-2), radiotherapy control system server(s) 1214 (1214-1 and 1214-2) and radiotherapy device(s) 1215 (the radiotherapy device located in the radiotherapy center 12-1 in FIG. 1 is marked with 1215-1 and the radiotherapy device located in the radiotherapy center 12-2 is marked with 1215-2) connected to the radiotherapy control system server(s) 1214.

The monitoring cloud platform 11 is also configured to control the target device(s) 121 according to the operating parameters of the target device(s) 121 and/or the patient treatment information generated by the target device(s) 121 in the radiotherapy center(s) 12.

Optionally, in order to enable a manager or a doctor of the radiotherapy center(s) 12 to call the operating parameters of all the target device(s) and the patient treatment information generated by the target device(s) that are generated before a current moment, the monitoring cloud platform 11 is further configured to store the operating parameters of the target device(s) 121 and/or the patient treatment information generated by the target device(s) 121 that are obtained.

Optionally, the monitoring cloud platform 11 being configured to control the target device(s) 121 according to the operating parameters of the target device(s) 121 in the radiotherapy center(s) 12 includes: the monitoring cloud platform 11 being configured to determine whether there is a failure of each target device 121 according to the operating parameters of the target device(s) 121 in the radiotherapy center(s) 12, wherein

if the monitoring cloud platform 11 determines that there are failure(s) of the target device(s) 121, the monitoring cloud platform 11 is further configured to send failure processing command(s) corresponding to the failure(s) to the radiotherapy center(s) 12 to cause the radiotherapy center(s) 12 to process the failure(s) of the target device(s) 121.

Optionally, the monitoring cloud platform 11 being configured to control the target device(s) 121 according to the patient treatment information generated by the target device(s) 121 in the radiotherapy center(s) 12 includes:

the monitoring cloud platform 11 being configured to determine whether the patient treatment information generated by the target device(s) 121 meets a preset treatment standard, the preset treatment standard being, for example, pre-stored treatment information, wherein

if the monitoring cloud platform 11 determines that the patient treatment information generated by the target device(s) 121 does not meet the preset treatment standard, the monitoring cloud platform 11 is further configured to send failure treatment command(s) to the radiotherapy center(s) 12 to cause the radiotherapy center(s) 12 to adjust operating state(s) of the target device(s) 121.

In order to enable the manager of the radiotherapy center(s) to clearly understand condition of the failure(s) of the radiotherapy center(s) and process the failure(s) of the radiotherapy center(s), optionally, in the case where the monitoring cloud platform determines that there are failure(s) of the target device(s), the monitoring cloud platform is further configured to send device failure prompt information to management terminal(s) corresponding to the target device(s).

The device failure prompt information at least includes one or more of the following: sound, text, and light.

In order to enable the manager of the radiotherapy center(s) to clearly understand condition of treatment failure(s) of the radiotherapy center(s) and process the treatment failure(s) of the radiotherapy center(s), optionally, in the case where the monitoring cloud platform determines that the patient treatment information generated by the target device(s) does not meet the preset treatment standard, the monitoring cloud platform is further configured to send treatment failure prompt information to the management terminal(s) corresponding to the target device(s).

The treatment failure prompt information at least includes one or more of the following: sound, text, and light.

For example, the device failure(s) at least include one or more of the following: the OIS server(s) being interrupted, calibration parameters in operating parameters of the TPS server(s) being different from TPS calibration parameters prestored in the monitoring cloud platform, geometric calibration parameters in operating parameters of the IGS server(s) being different from IGS geometric calibration parameters prestored in the monitoring cloud platform, configuration parameters in operating parameters of the radiotherapy control system server(s) being different from control configuration parameters prestored in the monitoring cloud platform, and treatment parameters in operating parameters of the radiotherapy device(s) being not within a preset range of radiotherapy treatment parameters prestored in the monitoring cloud platform.

The calibration parameters in the operating parameters of the TPS server(s) at least include a device dose rate.

The geometric calibration parameters in the operating parameters of the IGS server(s) at least include a relative position between a tube and a flat panel.

The configuration parameters in the operating parameters of the radiotherapy control system server(s) at least include an operation path of a treatment bed.

The treatment parameters in the operating parameters of the radiotherapy device(s) at least include: a serial number of a target point, a non-coplanar angle, a serial number of a collimator for treating the target point, irradiation time of the target point, and a rotation angle of a rack.

In the above embodiments, the monitoring cloud platform may obtain the operating parameters of the target device(s) in the radiotherapy center(s) and/or the patient treatment information generated by the target device(s) in real time, and may also control corresponding target device(s) according to the operating parameters and/or the patient treatment information generated by the target device(s) that are obtained. Therefore, the monitoring system for the radiotherapy center directly controls the corresponding target device(s) according to the operating parameters obtained, thereby monitoring all target devices in the radiotherapy center(s) and processes of treating all patients. Further, potential safety hazards or treatment errors may be discovered in time when occurring in the radiotherapy center(s), and corresponding remedial measures may be carried out.

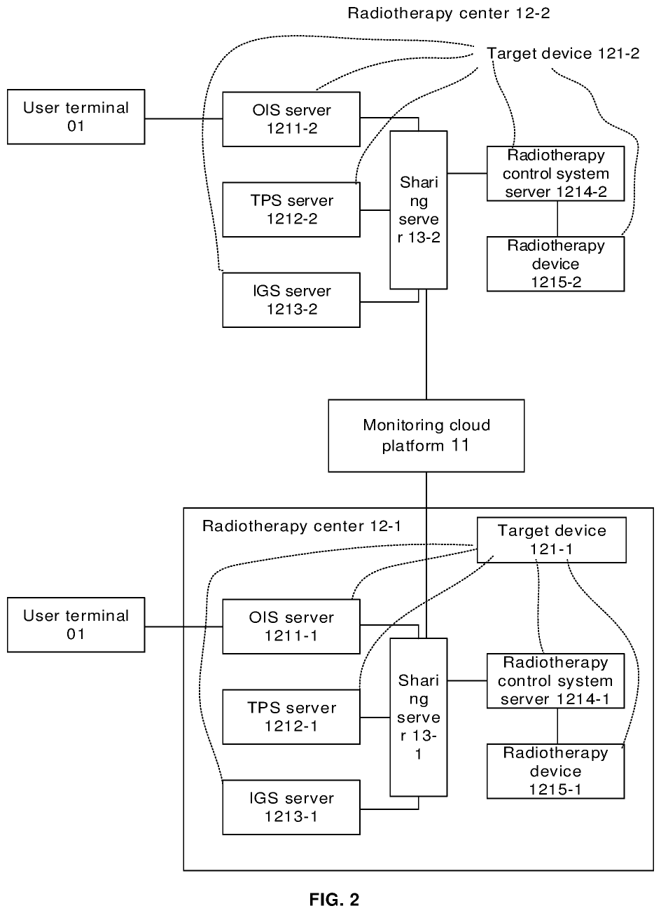

In practice, respective target devices in the radiotherapy center is independent. There is no unified information storage region for the manager or the doctor to monitor operations of all the target devices or a process of treating any patient in combination with all target device information. Therefore, referring to FIG. 2, the embodiments of the present disclosure further provide another monitoring system for the radiotherapy center, which further includes at least one sharing server 13 (two sharing server are taken as an example in FIG. 2, and the two sharing server are respectively marked with 13-1 and 13-2) on a basis of the foregoing embodiments.

The at least one sharing server 13 is disposed in the at least one radiotherapy center 12 and is in one-to-one correspondence with the at least one radiotherapy center 12. That is, a sharing server 13-1 corresponds to a radiotherapy center 12-1, and a sharing server 13-2 corresponds to a radiotherapy center 12-2.

The at least one sharing server 13 is connected to the target device(s) 121 of the radiotherapy center 12 in which the at least one sharing server 13 is located (FIG. 2 shows an example in which the radiotherapy centers include all the existing target devices), and is configured to obtain the operating parameters of the target device(s) 121 and the patient treatment information generated by the target device(s) 121.

The at least one sharing server 13 is further connected to the monitoring cloud platform 11, and is configured to send the operating parameters of the target device(s) 121 in the target device(s) 121 and the patient treatment information generated by the target device(s) 121 to the monitoring cloud platform 11.

For example, the operating parameters of the target device(s) at least include: operating parameters of the OIS server(s), the operating parameters of the TPS server(s), the operating parameters of the IGS server(s), the operating parameters of the radiotherapy control system server(s), and the operating parameters of the radiotherapy device(s).

The patient treatment information generated by the target device(s) at least includes: OIS treatment process information, TPS treatment process information, IGS treatment process information, control treatment process information, and radiotherapy treatment process information. For example, the information includes, but is not limited to, basic information of the patient (obtained by IGS), diagnosis information of the patient (obtained by IGS), prescription information of the patient (obtained by IGS), a radiotherapy plan (obtained by TPS), and implementation of the radiotherapy plan (obtained by the radiotherapy devices and the radiotherapy control system).

Optionally, referring to FIG. 2, each sharing server 13 (13-1 and 13-2) is connected to the OIS servers 1211 (1211-1 and 1211-2), the TPS servers 1212 (1212-1 and 1212-2), the IGS servers 1213 (1213-1 and 1213-2) and the radiotherapy control system servers 1214 (1214-1 and 1214-2) in the target devices 121 of the radiotherapy center 12 in which the sharing server 13 is located. The sharing server 13 is configured to obtain the operating parameters of the OIS servers and the OIS treatment process information generated by the OIS servers from the OIS servers 1211, obtain the operating parameters of the TPS servers and the TPS treatment process information generated by the TPS servers from the TPS servers 1213, obtain the operating parameters of the IGS servers and the IGS treatment process information generated by the IGS servers from the IGS servers 1213, obtain the operating parameters of the radiotherapy control system servers and the control treatment process information generated by the radiotherapy control system servers from the radiotherapy control system servers 1214, and obtain the operating parameters of the radiotherapy devices 1215 (1215-1 and 1215-2) and the radiotherapy treatment process information generated by the radiotherapy devices from the radiotherapy control system servers 1214.

In practice, family members of the patient or the patient himself or herself would also like to know treatment progress of the patient or treatment information during a treatment process when the family members of the patient or the patient himself or herself is not in the hospital. Therefore, optionally, as shown in FIG. 2, the monitoring system for the radiotherapy center further includes user terminal(s) 01 connected to the target device(s) 121 via a wireless network (FIG. 2 shows an example in which the OIS servers are connected to the user terminals).

The target device(s) 121 are configured to send target information corresponding to the query command(s) in the patient treatment information generated by the target device(s) 121 to the user terminal(s) 01 in response to query command(s) sent by the user terminal(s) 01.

After the connection relationship shown in FIG. 2 is completed, the monitoring system for the radiotherapy center provided by the embodiments of the present disclosure may achieve the following purposes.

(1) During an operation of the at least one radiotherapy device in each radiotherapy center, the operating parameters of the at least one radiotherapy device are sent to the sharing server through servers in the radiotherapy control system in real time, the sharing server uploads the operating parameters of the radiotherapy device in each radiotherapy center to the monitoring cloud platform approximately in real time, and the monitoring cloud platform monitors the operating parameters of the at least one radiotherapy device. In a case where operating parameters of a certain radiotherapy device (such as an accelerator or a gamma knife) are found to be abnormal currently, that is, in a case where the operating parameters of the certain radiotherapy device are not within a prestored range, it is indicated that there may be a failure of the radiotherapy device. In this case, the monitoring cloud platform sends, via the sharing server of the radiotherapy center in which the radiotherapy device is located, information indicating that the radiotherapy device is abnormal, i.e., the failure processing command in the foregoing embodiments, to a radiotherapy control system of the radiotherapy center. The servers in the radiotherapy control system immediately terminates the operation of the radiotherapy device, and the radiotherapy device is inspected and repaired in time.

(2) The OIS serves as a management system. The monitoring cloud platform monitors whether the OIS server is interrupted through an information interaction with a server in the OIS. If the OIS server is interrupted, a corresponding failure processing command is sent to the OIS server through the sharing server, so that the manager or the doctor checks and restarts the OIS server.

(3) The TPS uploads the calibration parameters (taking the device dose rate as an example) to the monitoring cloud platform for storage through the sharing server. In a generating process of the treatment plan, the monitoring cloud platform compares the calibration parameters obtained with the calibration parameters prestored in real time, and monitors whether the calibration parameters are changed or not in real time, thereby monitoring whether an operation of the TPS is normal. If the operation of the TPS is abnormal, a corresponding failure processing command is sent to the TPS server through the sharing server to cause the manager or the doctor to process the failure.

(4) The IGS uploads the geometric calibration parameters (such as the relative position between the tube and the flat panel) to the monitoring cloud platform through the sharing server for storage. The monitoring cloud platform compares the geometric calibration parameters obtained with the geometric calibration parameters prestored, and monitors whether the geometric calibration parameters are changed or not, thereby monitoring whether an operation of the IGS is normal. If the operation of the IGS is abnormal, a corresponding failure processing command is sent to the IGS server through the sharing server to cause the manager or the doctor to process the failure.

(5) The radiotherapy control system uploads the configuration parameters (such as the operation path of the treatment bed) to the monitoring cloud platform through the sharing server for storage. The monitoring cloud platform compares actual configuration parameters obtained by the radiotherapy control system with the configuration parameters prestored, and monitors whether the configuration parameters are changed or not, thereby monitoring whether an operation of the radiotherapy control system is normal. If the operation of the radiotherapy control system is abnormal, a corresponding failure processing command is sent to the radiotherapy control system server through the sharing server to cause the manager or the doctor to process the failure.

In addition, after the sharing server is provided in the radiotherapy center, a whole treatment process may be: when the patient arrives at a certain radiotherapy center, the IGS server(s) registers the basic information of the patient (at least including a patient's identification, such as an ID card number or a medical card number), and upload the basic information of the patient to the monitoring cloud platform via the sharing server. If an image of the patient is collected, the image information is also uploaded to the monitoring cloud platform via the sharing server. After the doctor makes a diagnosis, the doctor inputs the patient's diagnosis information through the IGS server(s), and the IGS server(s) upload the patient's diagnosis information to the monitoring cloud platform via the sharing server. And similarly, after the doctor makes a prescription for the patient according to the diagnosis information, the doctor inputs the patient's prescription information through the IGS server(s). The TPS server(s) may obtain the patient's image information and prescription information from the sharing server to make a treatment plan for the patient, and upload the treatment plan to the sharing server, and the sharing server sends the patient's information to the monitoring cloud platform. The radiotherapy control system obtains the patient's treatment plan from the sharing server through the patient's identification, and after the patient is fixed, the patient is accurately positioned though the IGS server(s). The IGS server(s) obtain reference image information (such as a CT image or a MRI image) in the patient's treatment plan from the sharing server, then collects a current actual position image of the patient, then registers the actual position image with the reference image to obtain a position offset, and sends the position offset to the radiotherapy control system. The radiotherapy control system controls an adjustment of a position of the radiotherapy device according to the position offset. After the patient is accurately positioned, the patient is treated (possibly in fractions) according to the patient's treatment plan. The radiotherapy device feeds back actual treatment parameters to the radiotherapy control system, and the radiotherapy control system compares the treatment parameters in the treatment plan with the actual treatment parameters. In a case where the treatment parameters in the treatment plan are inconsistent with the actual treatment parameters, the operation of the radiotherapy device is terminated, thereby ensuring an accuracy of radiotherapy. Herein, the treatment parameters may include the serial number of the target point, the non-coplanar angle, the serial number of the collimator for treating the target point, the irradiation time of the target point, and the rotation angle of the rack.

In practice, the patient may change a hospital during the treatment due to his or her own situation. Therefore, there is a need to enable different hospitals to find treatment information that is needed from the monitoring cloud platform after patient information provided by the patient is obtained. Therefore, optionally, as shown in FIG. 2:

the monitoring cloud platform 11 is configured to send target patient treatment information generated by target device(s) 121 in a second radiotherapy center 12-2 stored by the monitoring cloud platform 11 to the sharing server 13 provided in the first radiotherapy center 12-1 in response to a patient information search command sent by a sharing server 13 of a first radiotherapy center 12-1. The patient information search command carries a radiotherapy center identification and patient information.

According to the radiotherapy center identification and the patient information, if an identification of the second radiotherapy center 12-2 is matched with the radiotherapy center identification carried by the patient information search command, and the patient information included in the target patient treatment information is matched with the patient information carried by the patient information search command, the monitoring cloud platform 11 sends the target patient treatment information generated by the target devices 121 in the radiotherapy center 12-2 stored by the monitoring cloud platform 11 to the sharing server 13 in the radiotherapy center 12-1. In this way, whenever the patient changes the hospital (for example, when the image has been collected but no treatment plan is made), a hospital where the patient is currently located may find the patient's treatment progress and treatment process information from the monitoring cloud platform, and then make a subsequent treatment plan according to the patient's treatment progress and treatment process information that are obtained. Further, the doctor may also refer to previous treatment information of the patient to make a reasonable subsequent treatment. Different doctors may also learn related diagnosis and treatment experience from each other through the monitoring cloud platform.

Optionally, a manner in which the monitoring cloud platform, the sharing server(s) and the target device(s) or respective transceivers are connected includes at least one of the following: a WiFi network, a wired network, and bluetooth. The WiFi network may be a mobile operator network, and the above connection manner may also be a manner in which a plurality of network connection manners are combined.

In summary, in the monitoring system for the radiotherapy center provided by the embodiments of the present disclosure, the monitoring system includes the monitoring cloud platform. The monitoring cloud platform is connected to at least one radiotherapy center to obtain the operating parameters of the target device(s) in the radiotherapy center(s) and/or the patient treatment information generated by the target device(s) in real time. Then, the monitoring cloud platform controls the target device(s) according to the operating parameters of the target device(s) in the radiotherapy center(s). The monitoring cloud platform may obtain the operating parameters of the target device(s) in the radiotherapy center(s) and/or the patient treatment information generated by the target device(s) in real time, and may also control the corresponding target device(s) according to the operating parameters and/or the patient treatment information generated by the target device(s) that are obtained. Therefore, the monitoring system for the radiotherapy center directly controls the corresponding device(s) according to the operating parameters obtained, thereby monitoring all the devices of the radiotherapy center(s) and monitoring the processes of treating all the patients. Further, the potential safety hazards or the treatment errors may be discovered in time when occurring in the radiotherapy center(s), and the corresponding remedial measures may be carried out.

The foregoing descriptions are merely some specific implementation manners of the present disclosure, but the protection scope of the present disclosure is not limited thereto, and the changes or replacements that any person skilled in the art can easily think of in the technical scope disclosed by the present disclosure should be within the protection scope of the present disclosure. Therefore, the protection scope of the present disclosure shall be subjected to the protection scope of the claims.

1. A monitoring system for a radiotherapy center, the monitoring system comprising at least one radiotherapy center and a monitoring cloud platform, wherein
the monitoring cloud platform is connected to the at least one radiotherapy center, and is configured to obtain operating parameters of at least one target device in the at least one radiotherapy center in real time; and
the monitoring cloud platform is further configured to control the at least one target device according to the operating parameters of the at least one target device in the at least one radiotherapy center.

2. The monitoring system for the radiotherapy center according to claim 1, wherein the monitoring cloud platform is further configured to store the operating parameters of the at least one target device.

3. The monitoring system for the radiotherapy center according to claim 1, wherein the at least one radiotherapy center further includes at least one sharing server connected to the at least one target device and the monitoring cloud platform; and
the at least one sharing server is configured to obtain the operating parameters of the at least one target device in real time, and
to send the operating parameters of the at least one target device to the monitoring cloud platform.

4. The monitoring system for the radiotherapy center according to claim 1, wherein the monitoring cloud platform being further configured to control the at least one target device according to the operating parameters of the at least one target device in the at least one radiotherapy center includes:
the monitoring cloud platform being configured to determine whether there is a failure of the at least one target device according to the operating parameters of the at least one target device in the at least one radiotherapy center; and
the monitoring cloud platform being configured to send a failure processing command corresponding to the failure to the at least one radiotherapy center if the monitoring cloud platform determines that there is the failure of the at least one target device, wherein
the at least one radiotherapy center is configured to process the failure of the at least one target device in response to the failure processing command.

5. The monitoring system for the radiotherapy center according to claim 11, wherein the monitoring cloud platform being further configured to control the at least one target device according to the patient treatment information generated by the at least one target device in the at least one radiotherapy center includes:
the monitoring cloud platform being configured to determine whether the patient treatment information generated by the at least one target device meets a preset treatment standard; and
the monitoring cloud platform being configured to send a treatment failure command to the at least one radiotherapy center if the monitoring cloud platform determines that the patient treatment information generated by the at least one target device does not meet the preset treatment standard, wherein
the at least one radiotherapy center is configured to adjust an operating state of the at least one target device in response to the treatment failure command.

6. The monitoring system for the radiotherapy center according to claim 13, wherein the at least one radiotherapy center includes a first radiotherapy center and a second radiotherapy center;
the monitoring cloud platform is configured to send target patient treatment information generated by at least one target device in the second radiotherapy center stored by the monitoring cloud platform to a sharing server in the first radiotherapy center in response to a patient information search command send by the sharing server in the first radiotherapy center; and
an identification of the second radiotherapy center is matched with a radiotherapy center identification carried by the patient information search command, and patient information included in the target patient treatment information is matched with patient information carried by the patient information search command.

7. The monitoring system for the radiotherapy center according to claim 11, the monitoring system further comprising at least one user terminal connected to the at least one target device via a wireless network, wherein
the at least one target device is configured to send target information corresponding to a query command in the patient treatment information generated by the at least one target device to the at least one user terminal in response to the query command sent by at least one the user terminal.

8. The monitoring system for the radiotherapy center according to claim 4, wherein the at least one target device includes:
at least one of at least one tumor information system (OIS) server, at least one treatment planning system (TPS) server, at least one image guidance system (IGS) server, at least one radiotherapy control system server, and at least one radiotherapy device connected to the at least one radiotherapy control system server.

9. The monitoring system for the radiotherapy center according to claim 8, wherein the failure includes at least one of: the at least one OIS server being interrupted, calibration parameters in operating parameters of the at least one TPS server being different from TPS calibration parameters prestored in the monitoring cloud platform, geometric calibration parameters in operating parameters of the at least one IGS server being different from IGS geometric calibration parameters prestored in the monitoring cloud platform, configuration parameters in operating parameters of the at least one radiotherapy control system server being different from control configuration parameters prestored in the monitoring cloud platform, and treatment parameters in operating parameters of the at least one radiotherapy device being not within a preset range of radiotherapy treatment parameters prestored in the monitoring cloud platform.

10. The monitoring system for the radiotherapy center according to claim 3, wherein a manner in which the monitoring cloud platform, the at least one sharing server and the at least one target device are connected includes at least one of a WiFi network, a wired network and a bluetooth.

11. The monitoring system for the radiotherapy center according to claim 1, wherein the monitoring cloud platform is further configured to obtain patient treatment information generated by the at least one target device in real time, and to control the at least one target device according to the patient treatment information generated by the at least one target device in the at least one radiotherapy center.

12. The monitoring system for the radiotherapy center according to claim 11, wherein the monitoring cloud platform is further configured to store the patient treatment information generated by the at least one target device in the at least one radiotherapy center.

13. The monitoring system for the radiotherapy center according to claim 12, wherein the at least one radiotherapy center further includes at least one sharing server connected to the at least one target device in the at least one radiotherapy center and the monitoring cloud platform; and
the at least one sharing server is further configured to obtain the patient treatment information generated by the at least one target device in real time, and to send the patient treatment information generated by the at least one target device to the monitoring cloud platform.

14. A monitoring system for a radiotherapy center, the monitoring system comprising at least one radiotherapy center and a monitoring cloud platform, wherein
the monitoring cloud platform is connected to the at least one radiotherapy center, and is configured to obtain patient treatment information generated by at least one target device in the at least one radiotherapy center in real time; and
the monitoring cloud platform is further configured to control the at least one target device according to the patient treatment information generated by the at least one target device in the at least one radiotherapy center.

15. The monitoring system for the radiotherapy center according to claim 14, wherein the monitoring cloud platform is further configured to store the patient treatment information generated by the at least one target device in the at least one radiotherapy center.

16. The monitoring system for the radiotherapy center according to claim 14, wherein the at least one radiotherapy center further includes at least one sharing server connected to the at least one target device in the at least one radiotherapy center and the monitoring cloud platform; and
the at least one sharing server is configured to obtain the patient treatment information generated by the at least one target device in real time, and to send the patient treatment information generated by the at least one target device to the monitoring cloud platform.

17. The monitoring system for the radiotherapy center according to claim 14, wherein the monitoring cloud platform being further configured to control the at least one target device according to the patient treatment information generated by the at least one target device includes:
the monitoring cloud platform being configured to determine whether the patient treatment information generated by the at least one target device meets a preset treatment standard; and
the monitoring cloud platform being configured to send a treatment failure command to the at least one radiotherapy center if the monitoring cloud platform determines that the patient treatment information generated by the at least one target device does not meet the preset treatment standard, wherein
the at least one radiotherapy center is configured to adjust an operating state of the at least one target device in response to the treatment failure command.

18. The monitoring system for the radiotherapy center according to claim 16, wherein the at least one radiotherapy center includes a first radiotherapy center and a second radiotherapy center;
the monitoring cloud platform is further configured to send target patient treatment information generated by at least one target device in a second radiotherapy center stored by the monitoring cloud platform to a sharing server in the first radiotherapy center in response to a patient information search command sent by the sharing server in the first radiotherapy center, wherein
an identification of the second radiotherapy center is matched with a radiotherapy center identification carried by the patient information search command, and patient information included in the target patient treatment information is matched with patient information carried by the patient information search command.

19. The monitoring system for the radiotherapy center according to claim 14, the monitoring system further comprising at least one user terminal connected to the at least one target device via a wireless network, wherein
the at least one target device is configured to send target information corresponding to a query command in the patient treatment information generated by the at least one target device to the at least one user terminal in response to the query command sent by the at least one user terminal.

20. The monitoring system for the radiotherapy center according to claim 14, wherein the at least one target device includes at least one of at least one tumor information system (OIS) server, at least one treatment planning system (TPS) server, at least one image guidance system (IGS) server, at least one radiotherapy control system server, and at least one radiotherapy device connected to the at least one radiotherapy control system server.
